(19) United States
(12) Patent Application Publication
(10) Pub. No.: US 2007/0185069 A1
(43) Pub. Date: Aug. 9, 2007

(54) Anti-angiogenic activity of 2-methoxyestradiol in combination with anti-cancer agents

(76) Inventors: Stacy M. Plum, Arlington, VA (US); Steven J. Strawn, Arlington, VA (US); Theresa M. LaVallee, Rockville, MD (US); Carolyn F. Sidor, Chapel Hill, NC (US); William E. Fogler, Rockville, MD (US); Anthony M. Treston, Rockville, MD (US)

Correspondence Address: KING & SPALDING LLP, 1180 PEACHTREE STREET, ATLANTA, GA, 30309-3521, US

(21) Appl. No.: 11/599,997

(22) Filed: Nov. 14, 2006

Related U.S. Application Data
(60) Provisional application No. 60/736,220, filed on Nov. 14, 2005.
(60) Provisional application No. 60/788,354, filed on Mar. 31, 2006.

(51) Int. Cl. A61K 31/56

(52) U.S. Cl. 514/182; 514/506

(57) ABSTRACT

The present invention relates generally to methods and compositions of treating disease characterized by abnormal cell proliferation and/or abnormal or undesirable angiogenesis by administering antiangiogenic agents in combination with chemotherapeutic agents. More specifically, the present invention relates to a methods and compositions of treating diseases characterized by abnormal cell proliferation and/or abnormal or undesirable angiogenesis by administering 2-methoxyestradiol, in combination with chemotherapeutic agents.

### CROSS-REFERENCE TO RELATED APPLICATIONS

The present application claims the benefit of U.S. Provisional Patent Application Ser. No. 60/736,220, filed Nov. 14, 2005, and U.S. Provisional Patent Application Ser. No. 60/788,354, filed Mar. 31, 2006, which are incorporated herein by reference in their entirety.

### FIELD OF THE INVENTION

The present invention relates generally to compositions comprising anti-angiogenic agents in combination with anti-cancer agents and methods of use. More specifically, the present invention relates to methods and compositions of administering 2-methoxyestradiol with anti-cancer agents. More particularly, the present invention relates to methods of treating diseases characterized by abnormal cell proliferation and/or abnormal or undesirable angiogenesis by administering 2-methoxyestradiol in combination with anti-cancer agents.

### BACKGROUND OF THE INVENTION

The direct targeting of tumor cells by cytotoxic agents has been the main therapeutic strategy against advanced human malignant tumors. This strategy has achieved limited success in curing most cancer types, often only achieving temporary remission at the expense of negative systemic side effects. Several solid epithelial tumors are not sensitive to chemotherapy and there is an increasing problem in the development of drug resistance in tumors that are initially responsive to chemotherapy (Braverman, Am. Intern. Med. (1993); 118:630-32 and Gasparini et al. The Breast (1993); 2:27-32). In addition, there is a growing appreciation for the role the stroma, or non-tumor cells, play in determining the growth, proliferation and metastasis of a tumor. Angiogenesis, in particular, has been shown to play an important role in this regard.

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in many disease states, tumor metastases, and abnormal growth by endothelial cells. The diverse pathological disease states in which unregulated angiogenesis is present have been grouped together as angiogenic-dependent or angiogenic-associated diseases.

The hypothesis that tumor growth is angiogenesis-dependent was first proposed in 1971. (Folkman, New Eng. J Med., 285:1182-86 (1971)). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:

(1) The growth rate of tumors implanted in subcutaneous transparent chambers in mice is slow and linear before neovascularization, and rapid and nearly exponential after neovascularization. (Algire, et al., J. Nat. Cancer Inst., 6:73-85 (1945)).

(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, 164:491-502 (1966)).

(3) Tumor growth in the avascular cornea proceeds slowly and at a linear rate, but switches to exponential growth after neovascularization. (Gimbrone, Jr., et al., J. Nat. Cancer Inst., 52:421-27 (1974)).

(4) Tumors suspended in the aqueous fluid of the anterior chamber of a rabbit eye remain viable, avascular, and limited in size to <1 mm . Once they are implanted on the iris vascular bed, they become neovascularized and grow rapidly, reaching 16,000 times their original volume within 2 weeks. (Gimbrone, Jr., et al., J. Exp. Med., 136:261-76).

(5) When tumors are implanted on a chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, 35:347-56 (1977)).

(6) Vascular casts of metastases in a rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, 68:334-40 (1970)).

(7) In transgenic mice that develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, 339:58-61 (1989)).

(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, 362:841-44 (1993)).

(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 51:6180-84 (1991)).

(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumor cells in vitro. (Gross, et al., Proc. Am. Assoc. Cancer Res., 31:79 (1990)).

(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, 48:555-57 (1990)). There is also indirect clinical evidence that tumor growth is angiogenesis dependent.

(12) Human retinoblastomas that are metastatic to the vitreous develop into avascular spheroids that are restricted to less than 1 mm³ despite the fact that they are viable and incorporate ³H-thymidine (when removed from an enucleated eye and analyzed in vitro).

(13) Carcinoma of the ovary metastasizes to the peritoneal membrane as tiny avascular white seeds (1-3 mm³). These implants rarely grow larger until one or more of them become neovascularized.

(14) Intensity of neovascularization in breast cancer (Weidner, et al., New Eng. J. Med., 324:1-8 (1991); Weidner, et al., J. Nat. Cancer Inst., 84:1875-87 (1992)) and in prostate cancer (Weidner, et al., Am. J. Pathol., 143(2):401-09 (1993)) correlates highly with risk of future metastasis.

(15) Metastasis from human cutaneous melanoma is rare prior to neovascularization. The onset of neovascularization leads to increased thickness of the lesion and an increased risk of metastasis. (Srivastava, et al., Am. J. Pathol., 133:419-23 (1988)).

(16) In bladder cancer, the urinary level of an angiogenic protein, bFGF, is a more sensitive indicator of status and extent of disease than is cytology. (Nguyen, et al., J. Nat. Cancer Inst., 85:241-42 (1993)).

Thus, it is clear that angiogenesis plays a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis has been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

Several compounds have been used to inhibit angiogenesis. One such compound is 2-methoxyestradiol (2ME2). 2ME2 is a naturally occurring derivative of estradiol and has been shown to be an orally active, well-tolerated small molecule that possess anti-tumor and anti-angiogenic activity (Pribluda et al. Cancer Metastasis Rev. 19(1-2):173-9 (2000)). 2ME2 has low affinity for estrogen receptors, α and β, and its antiproliferative activity is independent of the interaction with those receptors (LaVallee et al. Cancer Research 62(13):3691-7 (2002)). Several mechanisms have been proposed for 2ME2 activity, including those mediated by its ability to bind to the colchicines binding site of tubulin (Cushman et al., 1995; D'Amato et al., 1994), destabilization of microtubules and inhibition of HIF-1α nuclear accumulation (Mabjeesh et al., 2003), induction of the extrinsic apoptotic pathway through upregulation of Death Receptor 5 (LaVallee et al. Cancer Research 63(2): 469-75 (2003)) and induction of the intrinsic apoptotic pathway, potentially through the inhibition of superoxide dismutase enzymatic activity (Huang et al. Trends Cell Biology 11(8):343-8, (2001)).

2ME2 has been shown to inhibit multiple mechanisms of progression in myeloma cells in vitro and in vivo and the ability to evade resistance mechanisms implicated in clinical resistance to conventional therapies (Dingli et al. Clinical Cancer Research, 8:3984-3954 (2002)). Inhibition of diverse tumor types including osteosarcoma (Maran et al. Bone (2002); 30:393-398 and Golebiewska et al. Act Biochim Pol (2002); 49:59-65), Ewing sarcoma (Djavaheri-Mergny et al. Oncogene (2003); 22:2558-67), pancreatic adenoma (Qanungo et al. Oncogene (2002); 21:4149-57 and Ryschich et al Pancreas (2003); 26:166-172), colon (Carothers et al. Cancer Lett (2002); 187:77-86), medulloblastoma (Kumar et al. Carcinogenesis (2003); 24:209-216), and melanoma (Ghosh et al. Melanoma Research (2003); 13:119-127) has also been demonstrated with 2ME2.

Anti-cancer therapy suffers from a number of limitations including, development of drug resistance, unwanted systemic side effects and limited efficacy against metastases. The use of combination therapy in order to overcome drug resistance, limit the unwanted side effects of certain anti-cancer agents, and improve their overall efficacy have been explored using various anti-cancer agent combinations. Finding safe and effective combination has not been easy. Combining anti-cancer agents has often led to combined toxicities or formulations containing dosages of limited use or efficacy. Therefore, there is a need to find formulations and methods of administering anti-cancer agents that can be combined safely and effectively without having to resort to dose-reduction of either agent, or in the case where dose-reduction is necessary or desired, the combination maintains efficacy by allowing for higher doses of the less toxic agent. 2-methoxyestradiol, with its multiple mechanisms of action, broad spectrum of inhibitory activity in a wide range of tumors, ability to overcome drug resistance in certain tumor types, and limited negative side effects make it a strong candidate for combination therapy. Previous studies have not looked at the ability to combine 2-methoxyestradiol and anti-cancer agents into a method of treating diseases characterized by abnormal cell proliferation and/or abnormal or undesirable angiogenesis. What is needed therefore, are novel compositions and methods of treating diseases characterized by abnormal cell proliferation (i.e. abnormal mitosis) and/or abnormal or undesirable angiogenesis comprising combination therapy involving 2-methoxyestradiol together with one or more anti-cancer agents.

Such compositions should be easy to administer and provide minimal or no side effects.

### SUMMARY OF THE INVENTION

The present invention comprises methods and compositions for treating disease characterized by abnormal cell proliferation and/or abnormal or undesirable angiogenesis comprising administering 2-methoxyestradiol in combination with anti-cancer agents. 2-methoxyestradiol is a powerful antiangiogenic and also has the ability to enhance the effects of other anti-cancer agents through its own anti-mitotic and pro-apoptotic capabilities. In one embodiment, the present invention comprises a method of treating diseases or conditions associated with or dependent on abnormal, undesirable and/or excessive angiogenesis and/or undesirable cell proliferation in a human or animal comprising administering to the human or animal an amount of a compound selected from one or more of the following; 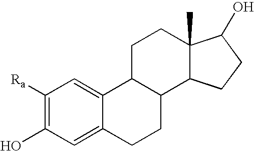
wherein Rₐ is selected from —OCH₃, —OCH₂CH₃, —CH₃, —CH₂CH₃, —CCCH₃, —CHCH—CH₃, or CH₂—CHCH₂ simultaneously with one or more anti-cancer agents.

In another embodiment, the present invention comprises a method of treating diseases or conditions associated with or dependent on abnormal, undesirable and/or excessive angiogenesis and/or undesirable cell proliferation in a human or animal comprising administering to the human or animal an amount of a compound selected from one or more of the following; 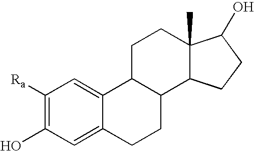
wherein Rₐ is selected from —OCH₃, —OCH₂CH₃, —CH₃, —CH₂CH₃, or —CCCH₃, CHCH—CH₃, or CH₂—CHCH₂ followed by administration of one or more anti-cancer agents.

In yet another embodiment, the present invention comprises a pharmaceutical preparation comprising a compound selected from one or more of the following; 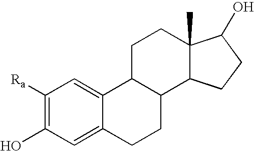
wherein Rₐ is selected from —OCH₃, —OCH₂CH₃, —CH₃, —CH₂CH₃, —CCCH₃, —CHCH—CH₃, or CH₂—CHCH₂ in combination with one or more anti-cancer agents. The pharmaceutical preparation can also comprise a pharmaceutically acceptable carrier, excipient or diluent.

Accordingly, it is an object of the present invention to provide a composition combining 2-methoxyestradiol in combination with one or more anti-cancer agents.

A further object of the present invention is to provide a method for administering 2-methoxyestradiol in combination with anti-cancer agents to mammals to more effectively inhibit angiogenesis and/or to more effectively treat diseases or conditions associated with undesirable and/or excessive angiogenesis and/or undesirable cell mitosis.

These and other objects, features and advantages of the present invention will become apparent after a review of the following detailed description of disclosed embodiments and the appended claims.

### BRIEF DESCRIPTION OF THE DRAWINGS

FIG. 1 shows the dose dependent inhibition of U87 MG tumor cell and HUVEC proliferation by 2ME2.

FIG. 2 shows the activity of 2ME2 or Temodar® (Schering Corp., Kenilworth, N.J.) against early stage U87 MG ectopic tumors.

FIGS. 3A and 3B shows activity of 2ME2 in combination with Temodar® (Schering Corp. Kenilworth, N.J.) against late stage U87 MG ectopic tumors.

FIG. 4 shows activity of 2ME2 in combination with Velcade® (Millennium, Cambridge, Mass.) in an RPMI-8226 xenograft model of myeloma

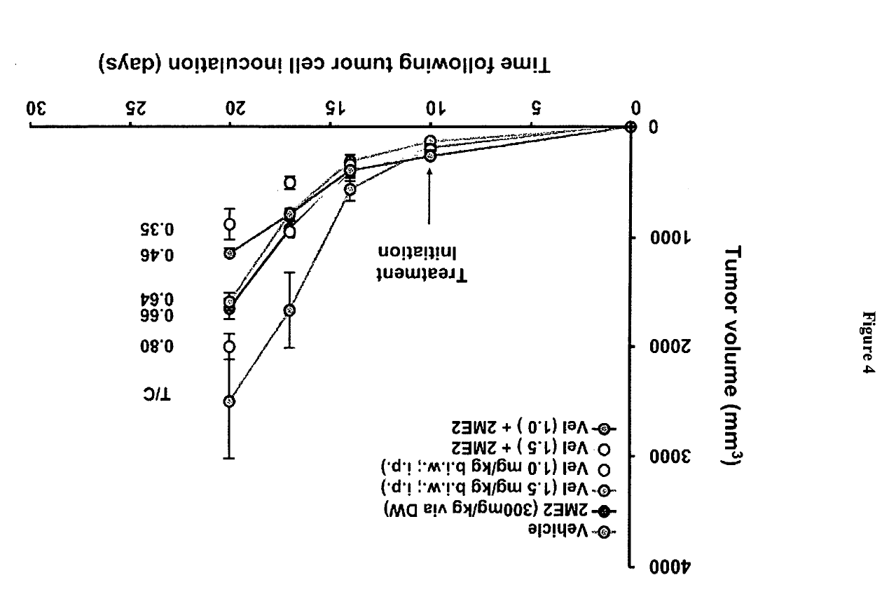

### DETAILED DESCRIPTION OF THE INVENTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated in their entireties by reference.

The present invention comprises methods and compositions for treating diseases or conditions associated with or dependent on abnormal, undesirable and/or excessive angiogenesis and/or undesirable cell proliferation comprising administering to a human or an animal 2-methoxyestradiol in combination with one or more anti-cancer agents.

2-methoxyestradiol

2-methoxyestradiol is an endogenous metabolite that is formed by the sequential hydroxylation of 17β-estradiol by cytochrome P450 followed by O-methylation by catechol-O-methytransferase. 2-methoxyestradiol inhibits growth and causes apoptosis in proliferating endothelial cells and cancer cells in vitro and has antitumor and antiangiogenic effects in vivo against several tumor types (Mooberry. Current Opinion in Oncology (2003); 15:425-430). In one embodiment, compounds are those of the general Formulae I: 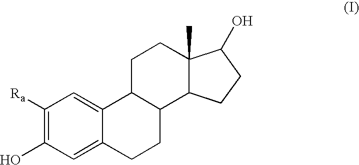
wherein Rₐ is selected from —OCH₃, —OCH₂CH₃, —CH₃, —CH₂CH₃, —CCCH₃, —CHCH—CH₃, or CH₂—CHCH₂. In cases where stereoisomers are possible, both R and S stereoisomers are envisioned as well as any mixture of stereoisomers.

Also included in this invention are prodrugs of the 2-methoxyestradiol and/or anti-cancer agents. Such prodrugs are produced using chemical procedures and synthetic routes well known to those of ordinary skill in the art. Strategies for creating prodrugs are well known to those skilled in the art and include amides, esters, ethers, thioesters, and thioethers of the agent. The chemical moieties linked to the agent can include naturally occurring and non-naturally occurring amino acids, sugars, peptides, low molecular weight organic moieties such as acetate and carbamate and benzoate and benzoyl, proteins including antibodies, and polymers including, but not limited to, polyethyleneglycols (PEGs). These moieties can be cleaved by enzymatic or non-enzymatic processes to generate the active anti-angiogenic agent. The characteristics of the prodrug are chosen to be useful for the desired purposes and routes of administration. For example, prodrugs containing amino acids can be used to enhance water solubility, prodrugs containing peptides can be used to enhance bioavailability and/or to enhance target tissue binding, and prodrugs containing PEGs can be used to increase plasma half-life.

Those skilled in the art will appreciate that the invention extends to other anti-cancer agents within the definitions given and in the claims below, having the described characteristics. These characteristics can be determined for each test compound using assays known in the art.

Anti-Cancer Agents

Anti-cancer agents that may be used with the following invention include, but are not limited to, chemotherapeutics, angiogenesis inhibitors, kinase inhibitors, histone deacetylase inhibitors as well as other modifiers of epigenetic pheomena and proteosome inhibitors.

Chemotherapeutic Agents

Chemotherapeutic agents are those compounds that non-specifically target rapidly dividing cells. They include alkylating agents, antimetabolites, anti-tumor antibiotics, mitotic inhibitors and nitrosureas. Representative chemotherapeutic agents that may be used in the instant invention include, but are not limited to, the following; Aldeskeukin, Alemtuzumab, alitretinoin, allopurinol, altretamine, amifostine, anastrozole, arsenic trioxide, asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B solution® (Orphan Medical Inc. Minnetonka, Minn.), epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, procarbazine, quinacrine Rasburicase, Rituximab, Sargramotim, streptozocin, talc, tamoxifen, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

Angiogenesis Inhibitors

Angiogenesis inhibitors are those compounds that prevent the growth of new capillary blood vessels from preexisting vessels. Angiogenesis inhibitors that may be used with present invention include both small-molecule and endogenous inhibitors of angiogenesis. Representative angiogenesis inhibitors that may be use with the present invention include, but are not limited to, the following; alpha interferon, angiogenic steroids, Bevacizumab, Batimastat (BB-94), Carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, beta interferon, gamma interferon, cartilage-derived inhibitor (CDI), gamma interferon inducibile protein (IP-10), gro-beta, heparinases, placental ribonuclease inhibitor, plasmingoen activator inhibitor, proliferen-related protein, retinoids, thrombospondin, TIMP-2, and 16 kd prolactin.

Kinase Inhibitors

Kinase inhibitors that may be used with the present invention include, but are not limited to, inhibitors of the following kinase families EGFR, HER2, VEGF receptor, FLT3, ABL, SRC, Janus (JAK), MTOR, Rho, cyclin dependent kinases (CDK), protein kinase C (PKC), phosphatidylinositol-3-kinase (PI3K), Aurora, MAP/MEK, Jun N-terminal (JNK). Representative kinase inhibitors that may be used with the following invention include, but are not limited to, cetuximab, Erlotinib, gefitinib, PKI-166, Canertinib (CI-1033), SU-11464, Matuzumab (Emd7200), EKB-569, Zd6474, Trastuzumab, GW-572016 (lapatinib ditosylate), PKC-412, Sutent, Vatalanib (Ptk787/ZK222584), CEP-701, SU5614, MLN518, XL999, VX-322, VEGF-trap, Imatinib mesylate, Azd0530, BMS-354825, SKI-606, CP-690, AG-490, WHI-P154, WHI-P131, Sirolimus, Everolimus, AP23573, Fasudil hydrochloride, Flavopiridol, Seliciclib (CYC202, roscovitine), SNS-032, Ruboxistaurin, Pkc412, Bryostatin, KAI-9803, SF1126, VX-680, Azd1152, Arry-142886 (AZD-6244), SCIO-469, GW681323 (SB-681323), CC-401, CEP-1347, Semaxanib (SU5416), Sunitinib (SU 11248) and Sorafenib (BAY 43-9006).

Histone Deacetylase Inhibitors

Histone deacetylase inhibitors that may be used with the following invention include, but are not limited to, AN-9 (butyric acid prodrug), sodium phenylbutrate, valproic acid, FK-228 (cyclic depsipeptide), MS-275 (benzamide), suberoylanilide hydroxamic acid and LAQ-824.

Proteosome Inhibitors

Proteasomes are enzymes with a complex structure and function. They are found abundantly in all cells, both normal and cancerous, and are responsible for the degradation of all regulatory proteins. Proteosome inhibitors include those compounds capable of inhibiting the assembly and/or function of these complexes. An example of a proteosome inhibitor that may be used with the present invention includes, but is not limited to, bortezomib.

Administration

In accordance with the present invention, the compounds of Formulae I may be mixed with one or more anti-cancer agents into a single formulation. The compounds of Formulae I and the anti-cancer agent may also be formulated and delivered separately.

The compositions described herein can be provided as physiologically acceptable formulations using known techniques, and the formulations can be administered by standard routes. In general, 2-methoxyestadiol and the anti-cancer agent can be administered by topical, oral, rectal or parenteral (e.g., intravenous, subcutaneous or intramuscular) route. In addition, the compositions can be incorporated into polymers allowing for sustained release, the polymers being implanted in the vicinity of where delivery is desired, for example, at the site of a tumor or within or near the eye, or the polymers can be implanted, for example, subcutaneously or intramuscularly or delivered intravenously or intraperitoneally to result in systemic delivery of 2-methoxyestradiol and/or anti-cancer agent. Other formulations for controlled, prolonged release of therapeutic agents useful in the present invention are disclosed in U.S. Pat. No. 6,706,289, the disclosure of which is incorporated herein by reference.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredient in an inert base such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. In one embodiment the topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration include particulate preparations of the anti-angiogenic agents, including, but not limited to, low-micron, or nanometer (e.g. less than 2000 nanometers, preferably less than 1000 nanometers, most preferably less than 500 nanometers in average cross section) sized particles, which particles are comprised of 2-methoxyestradiol and/or anti-cancer agent alone or in combination with accessory ingredients ort in a polymer for sustained release. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

In one embodiment, the compounds of Formulae I and the anti-cancer agent can be administered simultaneously. In another embodiment, they can be administered sequentially (i.e. 2ME2 dosage in the morning, anti-cancer agent dosage in the evening). Mixtures of more than one anti-cancer agent can, of course, be administered. Indeed, it is often desirable to use mixtures or sequential administrations of different anti-cancer agents to treat tumors, especially anti-cancer agents from the different classes.

If the 2-methoxyestradiol formulation and the anti-cancer agent are to be administered sequentially, the amount of time between administration of the 2-methoxyestradiol formulation and the anti-cancer agent will depend upon factors such as the amount of time it take the 2-methoxyestradiol formulation to be fully incorporated into the circulatory system of the host and the retention time of the 2-methoxyestradiol formulation in the host's body. In one embodiment, dosage formulations for 2-methoxyestradiol are disclosed in U.S. patent application Ser. No. 11/288,989, filed Nov. 29, 2005, which is incorporated herein by reference in its entirety.

The anti-cancer agent is administered in a therapeutically effective amount. This amount will be determined on an individual basis and will be based, at least in part, on consideration of the host's size, the specific disease to be treated, the severity of the symptoms to be treated, the results sought, and other such considerations. An effective amount can be determined by one of ordinary skill in the art employing such factors and using no more than routine experimentation.

It should be understood that, in addition to the ingredients particularly mentioned above, the formulations of the present invention may include other agents conventional in the art having regard to the type of formulation in question, for example, those suitable for oral administration may include flavoring agents, and nanoparticle formulations (e.g. less than 2000 nanometers, preferably less than 1000 nanometers, most preferably less than 500 nanometers in average cross section) may include one or more than one excipient chosen to prevent particle agglomeration.

Indications

The invention can be used to treat any disease characterized by abnormal cell proliferation and/or abnormal or undesirable angiogenesis. Such diseases include, but are not limited to, abnormal stimulation of endothelial cells (e.g., atherosclerosis); solid tumors; blood-borne tumors, such as leukemias; tumor metastasis; benign tumors, for example, hemangiomas, acoustic neuromas, neurofibromas, trachomas, and pyogenic granulomas; vascular malfunctions; abnormal wound healing; inflammatory and immune disorders; Bechet's disease; gout or gouty arthritis; abnormal angiogenesis accompanying: rheumatoid arthritis; skin diseases, such as psoriasis; diabetic retinopathy, and other ocular angiogenic diseases, such as retinopathy of prematurity (retrolental fibroplasia), macular degeneration, corneal graft rejection, neovascular glaucoma; liver diseases and Oster Webber Syndrome (Osler-Weber Rendu disease), epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, and corneal graph rejection.

Other diseases associated with neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

The present invention may also be used to treat angiogenesis-dependent cancers including, but not limited to, any one or combination of rhabdomyosarcoma, retinoblastoma, Ewing's sarcoma, neuroblastoma, and osteosarcoma. Other angiogenesis-dependent cancers treatable with the present invention include, but are not limited to, breast cancer, prostrate cancer, renal cell cancer, brain cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, non-Hodgkin's lymphoma, lip cancer, oral cancer, skin cancer, leukemia or multiple myeloma.

Pharmaceutical Preparations

Also contemplated by the present invention are implants or other devices comprised of the compounds or drugs of Formulae I, or prodrugs thereof, or other compounds included by reference where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., J. Neurosurg 74: pp. 441-446 (1991). These implants or devices can be implanted in the vicinity where delivery is desired, for example, at the site of a tumor or a stenosis, or can be introduced so as to result in systemic delivery of the agent.

Because anything not formed in the body as a natural component may elicit extreme and unexpected responses, such as blood vessel closure due to thrombus formation or spasm, and because damage to blood vessels by the act of insertion of a vascular stent may be extreme and unduly injurious to the blood vessel surface, it is prudent to protect against such events. Restenosis is a re-narrowing or blockage of an artery at the same site where treatment, such as an angioplasty or stent procedure, has already taken place. If restenosis occurs within a stent that has been placed in an artery, it is technically called “in-stent restenosis,” the end result being a narrowing in the artery caused by a build-up of substances that may eventually block the flow of blood. The compounds that are part of the present invention are especially useful to coat vascular stents to prevent restenosis. The coating should preferably be a biodegradable or non-biodegradable polymer that allows for a slow release of a compound of the present invention thereby preventing the restenosis event.

The present invention also relates to conjugated prodrugs and uses thereof. More particularly, the invention relates to conjugates of steroid compounds, such as compounds of Formulae I, and the use of such conjugates in the prophylaxis or treatment of conditions associated with enhanced angiogenesis or accelerated cell division, such as cancer, and inflammatory conditions, such as asthma and rheumatoid arthritis and hyperproliferative skin disorders including psoriasis. The invention also relates to compositions including the prodrugs of the present invention and methods of synthesizing the prodrugs.

In one aspect, the present invention provides a conjugated prodrug of an estradiol compound, preferably compounds of Formulae I, conjugated to a biological activity modifying agent.

Alternatively, the conjugated prodrug according to the present invention includes the compounds of Formulae I, conjugated to a peptide moiety.

The incorporation of an estradiol compound, such as the compounds of Formulae I, into a disease-dependently activated pro-drug enables significant improvement of potency and selectivity of this anti-cancer and anti-inflammatory agent.

A person skilled in the art will be able by reference to standard texts, such as Remington's Pharmaceutical Sciences 17th edition, to determine how the formulations are to be made and how these may be administered.

In a further aspect of the present invention there is provided use of compounds of Formulae I, or prodrugs thereof, in combination with a anti-cancer agent according to the present invention for the preparation of a medicament for the prophylaxis or treatment of conditions associated with angiogenesis or accelerated cell division or inflammation.

In a still further aspect of the present invention there is provided a method of prophylaxis or treatment of a condition associated with angiogenesis or accelerated or increased amounts of cell division, hypertrophic growth or inflammation, said method including administering to a patient in need of such prophylaxis or treatment an effective amount of compounds of Formulae I, or prodrugs thereof, in combination with an anti-cancer agent according to the present invention, as described herein. It should be understood that prophylaxis or treatment of said condition includes amelioration of said condition.

Pharmaceutically acceptable salts of the compounds of the Formulae I, or the prodrugs thereof, can be prepared in any conventional manner, for example from the free base and acid. In vivo hydrolysable esters, amides and carbamates and other acceptable prodrugs of Formulae I can be prepared in any conventional manner.

100% pure isomers are contemplated by this invention; however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. (1996) p. 3147-3176) and are well known to one skilled in the art. Such bioisosteric replacements include, for example, but are not limited to, substitution of ═S or ═NH for ═O.

A particularly useful formulation in the present invention is a nanoparticulate liquid suspension of 2-methoxyestradiol disclosed in U.S. patent application Ser. No. 10/392,403, filed Mar. 20, 2003 (the disclosure of which is incorporated herein by reference). This formulation is available from EntreMed, Inc., Rockville, Md., under the designation Panzem® NCD.

Known compounds that are used in accordance with the invention and precursors to novel compounds according to the invention can be purchased, e.g., from Sigma Chemical Co., Steraloids or Research Plus. Other compounds according to the invention can be synthesized according to known methods from publicly available precursors.

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### EXAMPLES

The data present in the Examples and the following table indicates that 2ME2 can be combined with a wide range of anti-cancer agents. The characteristics of 2ME2 and compounds of Formulae I are such that they can be combined with anti-cancer agents at the maximally tolerated or maximally effective dose and schedule of the anti-cancer agent. In some embodiments, combination with 2ME2 can be used to maintain the effectiveness while reducing the dose of the anti-cancer agent. Such reduction in dose can result in reduction of toxicity or reduction in any unacceptable effect or side-effect or the anti-cancer agent.

TABLE 1
Clinical Rationales from Tumor Models and Combinations

| | | |
|---|---|---|
| Model | Agent(s) | Activity |

| | | | |
|---|---|---|---|
| U87MG | Control | %Δ = | 416* |
| | Temodar ® | | 266 |
| | 2ME2 | | 333 |
| | Combined | | −33 |
| U87MG | Control | %Δ = | 1120 |
| | CPT-11 | | 670 |
| | 2ME2 | | 890 |
| | Combined | | 460 |
| RPMI-8226 | Velcade | T/C = | 0.64 |
| | 2ME2 | | 0.66 |
| | Combined | | 0.35 |
| LLC met | Paclitaxel | T/C = | 0.38 |
| | 2ME2 | | 0.37 |
| | Combined | | 0.05 |

| | | | |
|---|---|---|---|
| PC3 | XRT | T − C = | 2 d^{#} |
| | 2ME2 | | 0 d |
| | Combined | | 23 d |
| H2122 | Paclitaxel | MST = | 24 d |
| | 2ME2 | | 17 d |
| | Combined | | 35 d |

| | | | |
|---|---|---|---|
| LLC met | Cisplatin | T/C = | 0.19 |
| | 2ME2 | | 0.37 |
| | Combined | | 0.04 |
| CT-26 | 5-FU | T/C = | 0.38 |
| | 2ME2 | | 0.37 |
| | Combined | | 0.05 |

*Percent change in tumor volume from start of treatment
^{#}Difference in tumor doubling time

### Example 1

### Activity of 2ME2 in Combination with 5-Fluorouracil Against Syngenic Colon Carcinomas

2-methoxyestradiol (2ME2) is an endogenous metabolite of estradiol with antiproliferative, proapoptotic and antiangiogenic activity. Panzem® capsules have been evaluated in several Phase 1 and Phase 2 oncology clinical trials. A new formulation of 2ME2 with enhanced absorption, Panzem® NCD, is currently in clinical trials. In anticipation of Phase 2 clinical trials with Panzem® NCD, tests were conducted to assess its anti-tumor activity as either monotherapy or in combination with cytotoxic agents. In this preclinical study, the effectiveness of 2ME2 alone or with the antimetabolite, 5-Fluorouracil (5-FU) in the CT-26 syngeneic tumor model was evaluated. 5-FU is a well-recognized and commonly used antineoplastic agent used for treatment of colorectal cancer. Preliminary in vitro studies indicated that the IC50 for inhibition of proliferation with 2ME2 on CT-26 cells was 3.0 μM. Subsequent to these studies, male BALB/c mice were injected subcutaneously with 1×10⁶ CT-26 cells. Starting 7 days after tumor cell inoculation when mean tumor volume was approximately 100 mm³, cohorts of mice (n=5/group) began treatment with (a) vehicle alone; (b) 2ME@200mg/kg p.o. q.d.; (c) 5-FU 30mg/kg p.o. q.d.×5; (d) or a combination of 5-FU at 30 mg/kg p.o. q.d.×5 with 2ME2 at 50, 100, or 200 mg/kg p.o. q.d. This dose and regimen of 5-FU was determined to be the MTD in this model. Cohorts of mice receiving the combination of 5-FU and 2ME2 had oral administrations in the A.M. (5-FU) and the P.M. (2ME2). On study date 29, mean tumor volume in vehicle treated-control mice was 2494mm³+/−487. Treatment with either 2ME2 alone generated a T/C of 0.79 and 0.51. In comparison, 2ME2 at 50, 100, or 200 mg/kg, in combination with a 5-day cycle of 30mg/kg 5-FU, resulted in T/C values of 0.19, 0.25, and 0.16 respectively. Determination of mean changes in body weight as a consequence of treatment indicated moderate to no change with either 5-FU or 2ME2 administration. In contrast, mice treated with the combination demonstrated an unexpected decrease in body weight that was reversible with continued low dose 2ME2 treatment.

This study demonstrated that a combination strategy using 5-FU and 2ME2 can enhance the antitumor effectiveness of either agent alone in the treatment of a syngeneic colon carcinoma.

### Example 2

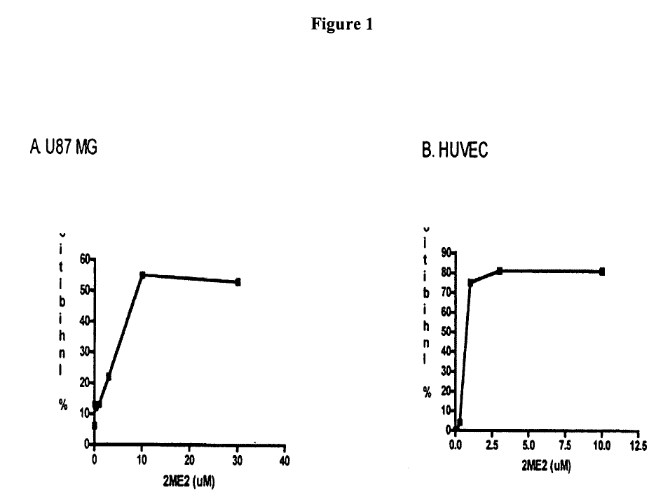

### Dose Dependent Inhibition of U87 MG Tumor Cells and HUVEC Proliferation by 2ME2 (FIG. 1)

U87 MG human glioblastoma cells were maintained in vitro in DMEM supplemented with 5% FBS, 2 mM glutamine, 1 mM sodium pyruvate, MEM vitamins and NEAA at 37° C. and 5% CO₂. HUVEC were maintained in M 200 media. For both HUVEC and U87 MG proliferation assays, cells were plated in a 96 well plate at 5×10³ cells per well and incubated at 37° C. overnight. At 24 hours, the media was aspirated and 2ME2 was administered to the cells at the following doses: 0.03, 0.1, 0.3, 1, 3, 10, 30 mM. Proliferation was assessed 48 hours after application of drug by WST-1(U87 MG) or BRDU (HUVEC).

Results: 2ME2 blocked cellular proliferation of both U87 MG cells and HUVEC in a dose dependent fashion. The IC50 value for inhibition of U87 MG proliferation is 2.4 mM. The IC50 value for HUVEC proliferation is 0.463 μM. The IC50 of Temodar® (Schering Corp. Kenilworth N.J.) was not determined since the agent requires in vivo activation to the active metabolite, 5-aminoimidazole-4 carboxamide. The IC50 value of 2ME2 against U87 MG in vitro helps determine an optimal dosage range for use with in vitro U87 MG glioblastoma xenograft model. The study also demonstrates that the dosage at which 2ME2 demonstrates an anti-proliferative effect directly on glioblastoma cells is nearly five times the dosage at which 2ME2 demonstrates an antiangiogenic effect on HUVEC. Therefore it is reasonable to assume that in vivo dosages needed to inhibit growth of the primary tumor should also be more than sufficient to inhibit angiogenesis in the surrounding stroma.

### Example 3

### Activity of 2ME2 or Temodar® Against Early Stage U87 MG Ectopic Tumors

Previous studies have shown that 2ME2 has antitumor activity in both ectopic and orthotopic glioma tumor models. To assess whether 2ME2 or Temodarg® (Schering Corp. Kenilworth, N.J.) impact tumor growth of U87 MG, we designed the following experiment. Male Balb/c SCID mice were injected subcutaneously with 1×10⁶ U87 MG tumor cells. 11 days following tumor cell inoculation when mean tumor volume was approximately 100 mm³, cohorts of mice began treatment with either vehicle control, 2ME2 400 or 200 mg/kg p.o., q.d. or Temodar® (Schering Corp. Kenilworth, N.J.) 42 mg/kg p.o., q.d.×5.

TABLE II
Study Design I

| Group | Dose | Treatment | No. of Mice |
|---|---|---|---|
| 1 | Vehicle Control | 0.2 ml p.o., q.d. | 10 |
| 2 | 2ME2 | 400 mg/kg p.o., q.d. | 10 |
| 3 | 2ME2 | 200 mg/kg p.o., q.d. | 10 |
| 4 | Temodar ® | 42 mg/kg p.o., q.d. × 5 | 10 |

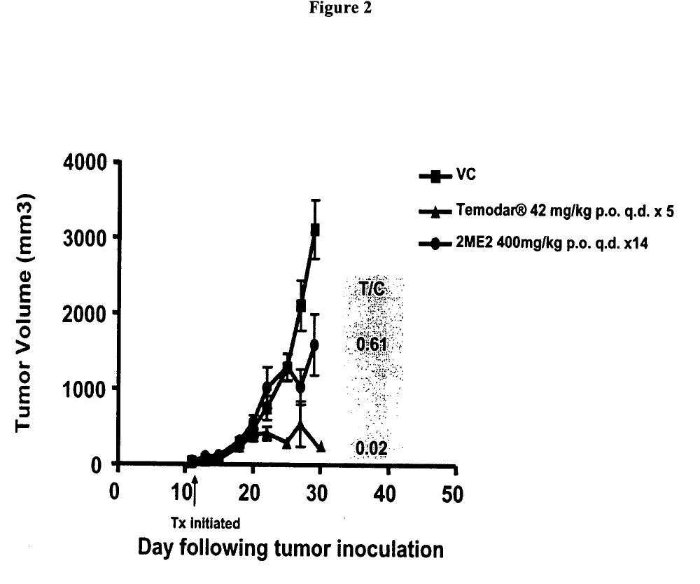

Results: On study day 30, the mean tumor volume of the vehicle control animals was approximately 3000 mm³. As shown in FIG. 2, 2ME2 at both 400 and 200 mg/kg inhibited tumor growth by approximately 40% (T/C =0.61). Using this dose and regimen in this model, Temodar® blocked U87 MG tumor growth by 98% (T/C =0.02). A separate tolerability study revealed this dose and regimen of Temodar® be the maximally tolerated dose.

The study demonstrates that both 2ME2 and Temodar® alone are effective against early stage glioblastomas and that Temodar® is more effective against early stage glioblastomas than 2ME2.

### Example 4

### Activity of 2ME2 in Combination with Temodar® Against Late Stage U87 MG Ectopic Tumors

In a second preclinical study the effectiveness of 2ME2 alone or in combination with Temodar® in the U87 MG glioblastoma xenograft tumor model was evaluated. Given the effectiveness of Temodar® at the maximally tolerated dose against 100 mm³ tumor burden, we altered the treatment strategy such that treatment was initiated when mean tumor volume was approximately 300 mm³. Cohorts of mice received treatment with either vehicle control, 2ME2 400 mg/kg p.o., q.d., Temodar® 42 mg/kg p.o., q.d×5, or 2ME2 400 mg/kg p.o., q.d. combined with Temodar® 42 mg/kg p.o., q.d.×5. Mice treated with the combination of 2ME2 and Temodar® received oral administration in the PM and AM, respectively.

TABLE III
Study Design II

| Group | Dose | Treatment | No. of Mice |
|---|---|---|---|
| 1 | Vehicle Control | 0.2 ml p.o., q.d. | 9 |
| 2 | 2ME2 | 400 mg/kg p.o., q.d. | 5 |
| 3 | Temodar ® | 42 mg/kg p.o., q.d. × 5 | 9 |
| 4 | 2ME2 + | 400 mg/kg p.o., q.d. + 42 | 9 |
| | Temodar ® | mg/kg p.o., q.d. × 5 | |

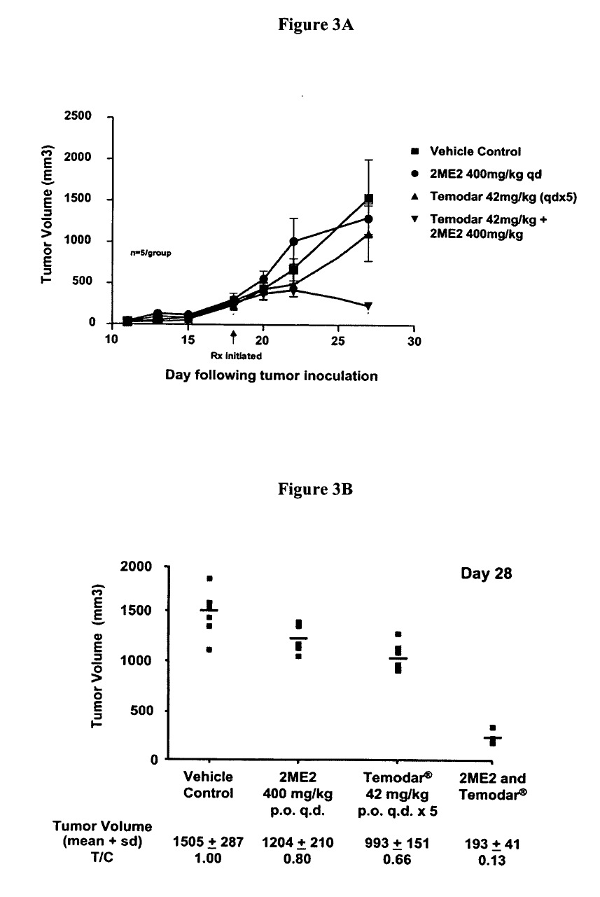

Results: On study day 28, mean tumor volume in the vehicle control animals was approximately 1500 mm³. As shown in FIGS. 3a and 3b, under these experimental conditions, treatment with either 2ME2 or Temodar® alone, generated a modest inhibition of tumor growth (20% and 34% inhibition respectively). In contrast, 2ME2 at 400 mg/kg in combination with a 5-day cycle of an MTD dose (42 mg/kg) of Temodar® resulted in 87% inhibition of tumor development.

This study demonstrates that the combination of 2ME2 and Temodar® is more effective at inhibiting and reducing the tumor size of late stage glioblastoma tumors than when either agent is administered alone.

We claim:

1. A method of treating diseases associated with undesirable and/or excessive angiogenesis and/or undesirable cell proliferation comprising administering simultaneously to an animal or human;
a) a first composition comprising a therapeutic agent selected from one or more of the following; 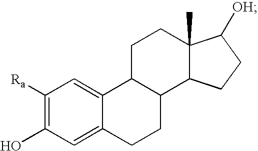
wherein Rₐ is selected from —OCH₃, —OCH₂CH₃, —CH₃, —CH₂CH₃, —CCCH₃, —CHCH—CH₃, or CH₂—CHCH₂ and a pharmaceutically acceptable carrier, diluent, or excipient; and
b) and one or more anti-cancer agents.

2. The method of claim 1, wherein the first composition and the one or more anti-cancer agents can be administered in a single formulation or in two or more separate formulations.

3. The method of claim 1, wherein the anti-cancer agent is a chemotherapeutic agent.

4. The method of claim 3, wherein the chemotherapeutic agent is selected from the following; Aldeskeukin, Alemtuzumab, alitretinoin, allopurinol, altretamine, amifostine, anastrozole, arsenic trioxide, Asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B solution®, epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, procarbazine, quinacrine Rasburicase, Rituximab, Sargramotim, streptozocin, talc, tamoxifen, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

5. The method of claim 1, wherein the anti-cancer agent is an angiogenesis inhibitor.

6. The method of claim 5, wherein the angiogenesis inhibitor is selected from one or more of the following; alpha interferon, angiogenic steroids, Bevacizumab Batimastat (BB-94), carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, beta interferon, gamma interferon, cartilage-derived inhibitor (CDI), gamma interferon inducibile protein (IP-10), gro-beta, heparinases, placental ribonuclease inhibitor, plasmingoen activator inhibitor, proliferen-related protein, retinoids, thrombospondin, TIMP-2, and 16 kd prolactin.

7. The method of claim 1, wherein the anti-cancer agent is a kinase inhibitor.

8. The method of claim 7, wherein the kinase inhibitor is selected from one or more of the following; cetuximab, Erlotinib, gefitinib, PKI-166, Canertinib (CI-1033), SU-11464, Matuzumab (Emd7200), EKB-569, Zd6474, Trastuzumab GW-572016 (lapatinib ditosylate), PKC-412, Sutent, Vatalanib (Ptk787/ZK222584), CEP-701, SU5614, MLN518, XL999, VX-322, VEGF-trap, Imatinib mesylate, Azd0530, BMS-354825, SKI-606, CP-690, AG-490, WHI-P154, WHI-P131, Sirolimus, Everolimus, AP23573, Fasudil hydrochloride, Flavopiridol, Seliciclib (CYC202, roscovitine), SNS-032, Ruboxistaurin, Pkc412, Bryostatin, KAI-9803, SF1126, VX-680, Azd1152, Arry-142886 (AZD-6244), SCIO-469, GW681323 (SB-681323), CC-401, CEP-1347, Semaxanib (SU5416), Sunitinib (SU11248) and Sorafenib (BAY 43-9006).

9. The method of claim 1, wherein the anti-cancer agent is an histone deacetylase inhibitor.

10. The method of claim 9, wherein the histone deacetylase inhibitor is selected from on or more of the following; AN-9 (butyric acid prodrug), sodium phenylbutrate, valproic acid, FK-228 (cyclic depsipeptide), MS-275 (benzamide), suberoylanilide hydroxamic acid and LAQ-824.

11. The method of claim 1, wherein the anti-cancer agent is a proteosome inhibitor.

12. The method of claim 1 1, wherein the proteosome inhibitor is bortezomib.

13. The method of claim 2, wherein the administration of the single formulation is oral, parenteral, transdermal, topical, intravenous, subcutaneous, intramuscular, intradermal, ophthalmic, epidural, intratracheal, sublingual, buccal, rectal, vaginal, nasal or inhalation.

14. The method of claim 2, wherein the administration of the separate formulations is oral, parenteral, transdermal, topical, intravenous, subcutaneous, intramuscular, intradermal, ophthalmic, epidural, intratracheal, sublingual, buccal, rectal, vaginal, nasal or inhalation.

15. The method of claim 1, wherein the first composition further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

16. The method of claim 2, wherein the single formulation is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

17. The method of claim 2, wherein the separate formulations are administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

18. The method of claim 1, wherein the angiogenesis is associated with diabetic retinopathy, retinopathy of prematurity, corneal graft rejection, neovascular glaucoma, retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, arthritis, rheumatoid arthritis, polyarteritis, systemic lupus, Wegener's sarcoidosis, scleritis, Stevens-Johnson disease, radial keratotomy, macular degeneration, sickle cell anemia, sarcoid, pseudoxanthoma elasticum, Paget's disease, vein occlusion, artery occlusion, carotid obstructive disease, chronic uveitis, chronic vitritis, Lyme's disease, Eales' disease, Behcet's disease, myopia, optic pits, Stargardt's disease, pars planitis, chronic retinal detachment, hyperviscosity syndromes, toxoplasmosis, post-laser complications, abnormal proliferation of fibrovascular or fibrous tissue, hemangiomas, Osler-Weber-Rendu disease, solid tumors, blood-borne tumors, acquired immune deficiency syndrome, ocular neovascular disease, age-related macular degeneration, osteoarthritis, diseases caused by chronic inflammation, Crohn's disease, ulcerative colitis, tumors of rhabdomyosarcoma, tumors of retinoblastoma, Ewing's sarcoma, neuroblastoma, tumors of osteosarcoma, leukemia, psoriasis, atherosclerosis, pemphigoid, infections causing retinitis, infections causing choroiditis, presumed ocular histoplasmosis, Best's disease, proliferative vitreoretinopathy, Bartonellosis, acoustic neuromas, neurofibroma, trachoma, pyogenic granulomas, vascular malfunctions, abnormal wound healing, gout or gouty arthritis, angiogenesis-dependent cancer, hereditary hemorrhagic telangiectasia, post-menopausal symptoms, osteoporosis, cardiovascular disease, myocardial angiogenesis, plaque neovascularization, hemophiliac joints, angiofibroma, wound granulation, intestinal adhesions, scleroderma, keloids, endometriosis.

19. The method of claim 1, wherein the angiogenesis is associated with angiogenesis-dependent cancers selected from breast cancer, prostrate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple myeloma.

20. The method of claim 19, wherein the cancer is colon cancer.

21. The method of claim 19, wherein the cancer is ectopic or orthotopic glioma.

22. The method of claim 19, wherein the cancer is multiple myeloma.

23. A method for treating diseases associated with undesirable and/or excessive angiogenesis and/or undesirable cell proliferation comprising administering sequentially to an animal or human;
a) a first composition comprising, a therapeutic agent selected from one or more of the following 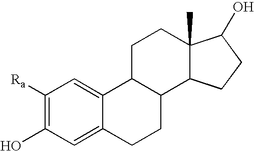
wherein Rₐ is selected from —OCH₃, —OCH₂CH₃, —CH₃, —CH₂CH₃, —CCCH₃, —CHCH—CH₃, or CH₂—CHCH₂ and a pharmaceutically acceptable carrier, diluent, or excipient; and
b) a second composition comprising one or more anti-cancer agents.

24. The method of claim 23, wherein the anti-cancer agent is a chemotherapeutic agent.

25. The method of claim 24, wherein the chemotherapeutic agent is selected from the following; Aldeskeukin, Alemtuzumab, alitretinoin, allopurinol, altretamine, amifostine, anastrozole, arsenic trioxide, Asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B solution®, epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, procarbazine, quinacrine Rasburicase, Rituximab, Sargramotim, streptozocin, talc, tamoxifen, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

26. The method of claim 23, wherein the anti-cancer agent is an angiogenesis inhibitor.

27. The method of claim 26, wherein the angiogenesis inhibitor is selected from one or more of the following; Bevacizumab, alpha interferon, angiogenic steroids, Batimastat (BB-94), carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, beta interferon, gamma interferon, cartilage-derived inhibitor (CDI), gamma interferon inducibile protein (IP-10), gro-beta, heparinases, placental ribonuclease inhibitor, plasmingoen activator inhibitor, proliferen-related protein, retinoids, thrombospondin, TIMP-2, and 16 kd prolactin.

28. The method of claim 23, wherein the anti-cancer agent is a kinase inhibitor.

29. The method of claim 28, wherein the kinase inhibitor is selected from one or more of the following; cetuximab, Erlotinib, gefitinib, PKI-166, Canertinib (CI-1033), SU-11464, Matuzumab (Emd7200), EKB-569, Zd6474, Trastuzumab, GW-572016 (lapatinib ditosylate), PKC-412, Sutent, Vatalanib (Ptk787/ZK222584), CEP-701, SU5614, MLN518, XL999, VX-322, VEGF-trap, Imatinib mesylate, Azd0530, BMS-354825, SKI-606, CP-690, AG-490, WHI-P154, WHI-P131, Sirolimus, Everolimus, AP23573, Fasudil hydrochloride, Flavopiridol, Seliciclib (CYC202, roscovitine), SNS-032, Ruboxistaurin, Pkc412, Bryostatin, KAI-9803, SF1126, VX-680, Azd1152, Arry-142886 (AZD-6244), SCIO-469, GW681323 (SB-681323), CC-401, CEP-1347, Semaxanib (SU5416), Sunitinib (SU11248) and Sorafenib (BAY 43-9006).

30. The method of claim 23, wherein the anti-cancer agent is an histone deacetylase inhibitor.

31. The method of claim 30, wherein the histone deacetylase inhibitor is selected from on or more of the following; AN-9 (butyric acid prodrug), sodium phenylbutrate, valproic acid, FK-228 (cyclic depsipeptide), MS-275 (benzamide), suberoylanilide hydroxamic acid and LAQ-824.

32. The method of claim 23, wherein the anti-cancer agent is a proteosome inhibitor.

33. The method of claim 32, wherein the proteosome inhibitor is bortezomib.

34. The method of claim 23, wherein the administration of the first composition is oral, parenteral, transdermal, topical, intravenous, subcutaneous, intramuscular, intradermal, ophthalmic, epidural, intratracheal, sublingual, buccal, rectal, vaginal, nasal or inhalation.

35. The method of claim 23, wherein the administration of the second composition is oral, parenteral, transdermal, topical, intravenous, subcutaneous, intramuscular, intradermal, ophthalmic, epidural, intratracheal, sublingual, buccal, rectal, vaginal, nasal or inhalation.

36. The method of claim 23, wherein the first composition further comprises an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

37. The method of claim 23, wherein the first composition is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

38. The method of claim 23, wherein the administration of the second composition is oral, parenteral, transdermal, intravenous, subcutaneous, intramuscular, intradernal, epidural, or intratracheal, sublingual, buccual, rectal, vaginal, nasal or inhalation.

39. The method of claim 23, wherein the second composition is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

40. The method of claim 23, wherein the angiogenesis is associated with diabetic retinopathy, retinopathy of prematurity, corneal graft rejection, neovascular glaucoma, retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, arthritis, rheumatoid arthritis, polyarteritis, systemic lupus, Wegener's sarcoidosis, scleritis, Stevens-Johnson disease, radial keratotomy, macular degeneration, sickle cell anemia, sarcoid, pseudoxanthoma elasticum, Paget's disease, vein occlusion, artery occlusion, carotid obstructive disease, chronic uveitis, chronic vitritis, Lyme's disease, Eales' disease, Behcet's disease, myopia, optic pits, Stargardt's disease, pars planitis, chronic retinal detachment, hyperviscosity syndromes, toxoplasmosis, post-laser complications, abnormal proliferation of fibrovascular or fibrous tissue, hemangiomas, Osler-Weber-Rendu disease, solid tumors, blood-borne tumors, acquired immune deficiency syndrome, ocular neovascular disease, age-related macular degeneration, osteoarthritis, diseases caused by chronic inflammation, Crohn's disease, ulcerative colitis, tumors of rhabdomyosarcoma, tumors of retinoblastoma, Ewing's sarcoma, neuroblastoma, tumors of osteosarcoma, leukemia, psoriasis, atherosclerosis, pemphigoid, infections causing retinitis, infections causing choroiditis, presumed ocular histoplasmosis, Best's disease, proliferative vitreoretinopathy, Bartonellosis, acoustic neuromas, neurofibroma, trachoma, pyogenic granulomas, vascular malfunctions, abnormal wound healing, gout or gouty arthritis, angiogenesis-dependent cancer, hereditary hemorrhagic telangiectasia, post-menopausal symptoms, osteoporosis, cardiovascular disease, myocardial angiogenesis, plaque neovascularization, hemophiliac joints, angiofibroma, wound granulation, intestinal adhesions, scleroderma, keloids, endometriosis.

41. The method of claim 23, wherein the angiogenesis is associated with angiogenesis-dependent cancers selected from breast cancer, prostrate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple myeloma.

42. The method of claim 41, wherein the cancer is colon cancer.

43. The method of claim 41, wherein the cancer is ectopic or orthotopic glioma.

44. The method of claim 41, wherein the cancer is multiple myeloma.

45. A composition for treating diseases associated with undesirable and/or excessive angiogenesis and/or undesirable cell proliferation comprising;
a) a compound selected from one or more of the following; 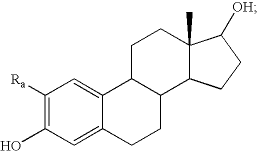
wherein Rₐ is selected from —OCH₃, —OCH₂CH₃, —CH₃, —CH₂CH₃, —CCCH₃, —CHCH—CH₃, or CH₂—CHCH₂;
b) one or more anti-cancer agents; and
c) a pharmaceutically acceptable carrier, diluent, or excipient.

46. The composition of claim 45, wherein the anti-cancer agent is a chemotherapeutic.

47. The composition of claim 46, wherein the chemotherapeutic agent is selected from the following; Aldeskeukin, Alemtuzumab, alitretinoin, allopurinol, altretamine, amifostine, anastrozole, arsenic trioxide, Asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B solution®, epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, procarbazine, quinacrine Rasburicase, Rituximab, Sargramotim, streptozocin, talc, tamoxifen, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

48. The composition of claim 45, wherein the anti-cancer agent is an angiogenesis inhibitor.

49. The composition of claim 48, wherein the angiogenesis inhibitor is selected from one or more of the following; Bevacizumab, alpha interferon, angiogenic steroids, Batimastat (BB-94), carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, beta interferon, gamma interferon, cartilage-derived inhibitor (CDI), gamma interferon inducibile protein (IP-10), gro-beta, heparinases, placental ribonuclease inhibitor, plasmingoen activator inhibitor, proliferen -related protein, retinoids, thrombospondin, TIMP-2, and 16kd prolactin.

50. The composition of claim 45, wherein the anti-cancer agent is a kinase inhibitor.

51. The composition of claim 50, wherein the kinase inhibitor is selected from one or more of the following; cetuximab, Erlotinib, gefitinib, PKI-166, Canertinib (CI-1033), SU-11464, Matuzumab (Emd7200), EKB-569, Zd6474, Trastuzumab®, GW-572016 (lapatinib ditosylate), PKC-412, Sutent, Vatalanib (Ptk787/ZK222584), CEP-701, SU5614, MLN518, XL999, VX-322, VEGF-trap, Imatinib mesylate, Azd0530, BMS-354825, SKI-606, CP-690, AG-490, WHI-P154, WHI-P131, Sirolimus, Everolimus, AP23573, Fasudil hydrochloride, Flavopiridol, Seliciclib (CYC202, roscovitine), SNS-032, Ruboxistaurin, Pkc412, Bryostatin, KAI-9803, SF1126, VX-680, Azd 1152, Arry-142886 (AZD-6244), SCIO-469, GW681323 (SB-681323), CC-401, CEP-1347, Semaxanib (SU5416), Sunitinib (SU11248) and Sorafenib (BAY 43-9006).

52. The composition of claim 45, wherein the anti-cancer agent is an histone deacetylase inhibitor.

53. The composition of claim 52, wherein the histone deacetylase inhibitor is selected from on or more of the following; AN-9 (butyric acid prodrug), sodium phenylbutrate, valproic acid, FK-228 (cyclic depsipeptide), MS-275 (benzamide), suberoylanilide hydroxamic acid and LAQ-824.

54. The composition of claim 45, wherein the anti-cancer agent is a proteosome inhibitor.

55. The composition of claim 54, wherein the proteosome inhibitor is bortezomib.
